(19) United States
(12) Patent Application Publication
(10) Pub. No.: US 2026/0137762 A1
(43) Pub. Date: May 21, 2026

(54) Method of Chemically Loosening Adhesions and Kit for Same

(71) Applicant: Hakala Medical Inc., San Diego, CA (US)

(72) Inventor: Neta Glaser, San Diego, CA (US)

(73) Assignee: Hakala Medical Inc., San Diego, CA (US)

(21) Appl. No.: 19/394,805

(22) Filed: Nov. 19, 2025

Related U.S. Application Data
(60) Provisional application No. 63/723,074, filed on Nov. 20, 2024.

(51) Int. Cl. A61K 38/48

(52) U.S. Cl. CPC A61K 38/4886

(57) ABSTRACT

A surgical procedure comprising selectively delivering a chemical adhesiolysis material to a treatment area of an adhesion bound to adjoining tissue in a body of a patient. The procedure includes waiting a period of time for the chemical adhesiolysis material to sufficiently loosen the adhesion. The lysing of the adhesion with the chemical adhesiolysis material permits access to a surgical site in order to perform the surgical procedure and reduces the risk of damage to the adjoining tissue in the body. A kit can optionally be provided.

### CROSS REFERENCE TO RELATED APPLICATION

This application claims the benefit of U.S. provisional patent application Serial No. 63/723,074 filed Nov. 20, 2024, the entire content of which is incorporated herein by this reference.

### FIELD OF THE INVENTION

The present invention relates to the field of the medical field and, more specifically, to the treatment and breakdown of adhesions.

### BACKGROUND OF THE INVENTION

Adhesions form inside a mammalian body after tissues experience trauma, for example from chemotherapy, previous surgery, infection or an immune response. They are made out of fibrin and collagen, and often bind tissues that are not supposed to be connected to each other, for example organs to other organs, to omentum or to an abdominal wall or different loops of bowel adhering to each other. Adhesions vary in grade, from filmy adhesions that can easily be separated (see FIG. 1), to dense, vascularized adhesions that require sharp dissection to break down (see FIG. 2). Patients typically have multiple grades of adhesions in their body. Adhesions can be found in any part of the body, for example between organs, in joints or in an eye, that experienced previous trauma, for example injury, chemotherapy, infection, surgery or an immune response. Adhesions can cause complications, for example small bowel obstruction (SBO), infertility, chronic pain or glaucoma.

Patients sometimes need surgery in areas that contain adhesions, for example to remove existing adhesions or treat an unrelated condition. When patients undergo surgery in an area affected by adhesions, the adhesions can make it difficult for the surgeon to manipulate the tissues in order to locate the surgical site. The surgeon may need to first break apart the adhesions, in a process that is called adhesiolysis, before being able to start the intended surgery. When surgeons perform adhesiolysis, they often begin by lysing the filmiest adhesions and work their way up the grades of adhesions, ending by performing adhesiolysis on the densest adhesions. A filmier adhesion, for example as shown in FIG. 1 with the assistance of a tool, can be broken apart with a blunt object in a laparoscopic procedure or a gloved hand in an open procedure. A denser adhesion, for example as shown in FIG. 2 within the dot-dash lined enclosure, can often be broken down with adhesiolysis tools. Standard of care (SoC) adhesiolysis can be performed mechanically with a blade, for example scissors (see FIG. 3), or electrically, for example with an electric scalpel.

Adhesiolysis can increase the complexity of a surgery. For example, adhesiolysis can add procedure time, which can increase the time that a patient is under anesthesia and thus risks related to anesthesia. Adhesiolysis can typically take an average of 20 minutes for lower grade adhesions and can take more than 3 hours for more dense and vascularized adhesions. Mechanical and electrical adhesiolysis of more dense adhesions can increase the risk of injuries, for example accidentally perforating a bowel wall or penetrating the intestine. Injuries to adjacent tissues occur in about 19-39% of procedures involving adhesiolysis and adhesiolysis complications result in about 2.3 billion dollars in direct surgical costs per year in the U.S. alone. These complications can result in infection, sepsis and death. There is currently no chemical solution for performing adhesiolysis.

Collagenase, an enzyme that breaks down collagen, is approved by the FDA for several applications related to chemically loosening or dissolving tissues. FDA-approved commercialized collagenase products include a petroleum-based cream that is used to treat diabetic foot ulcers and liquid injections that are used to treat cellulite and diseases related to excess collagen buildup in the body, for example Peyronie’s disease and Dupuytren’s contracture. However, neither of the existing products are appropriate for use in a surgical setting. Petroleum traps bacteria under it, increasing the risk of infections. It is carcinogenic when applied directly to internal organs, and is highly flammable in oxygen-rich environments like operating rooms. A liquid injection may disadvantageously flow away from the injection site and have unintended effects on other tissues. Additionally, the treatment regime of the existing liquid collagenase injections involves a series of injections over several weeks, which would not make sense with the procedural time constraints within an operating room (OR).

There is a need for improved methods of lysing adhesions that reduce injuries to the patients and improve outcomes for the healthcare system as a whole.

### BRIEF DESCRIPTION OF THE DRAWINGS

The drawings herein are all schematic and not drawn to scale. The drawings are for illustration purposes only and are not intended to limit the scope of the present disclosure.

FIG. 1. is a photograph that includes a thin, filmy adhesion in a mammalian body.

FIG. 2. is a photograph that includes a dense, vascularized adhesion in a mammalian body.

FIG. 3. is an example of current SoC adhesiolysis of an adhesion in a mammalian body in which the adhesion is being lysed with robotic scissors in a laparoscopic procedure.

FIG. 4. is a drawing of a dense adhesion connecting the bowel to the peritoneum in a mammalian body.

FIG. 5 is an embodiment of a step of the method of the invention in which a surgeon applies a chemical adhesiolysis aid of the invention, for example in the form of a gel, directly to a portion of the adhesion.

FIG. 6 is a drawing of a step of the method of FIG. 5 in which the chemical adhesiolysis aid acts on the adhesion and loosens it. The surgeon can optionally use a blunt tool, for example a surgical probe, to further break down the adhesion.

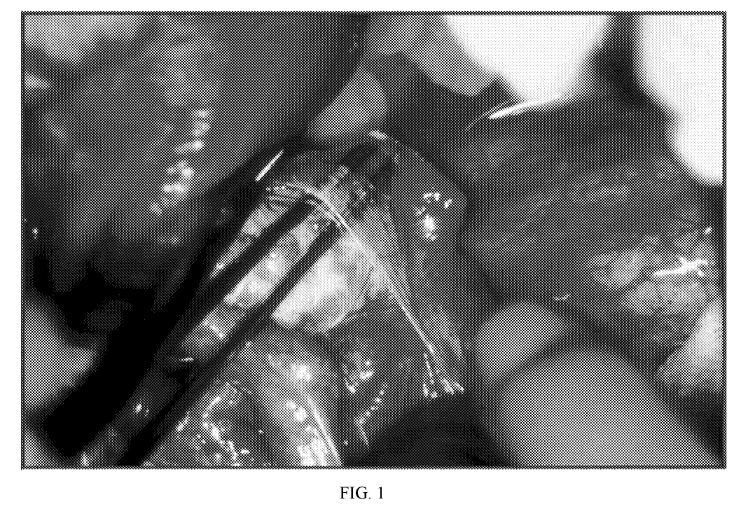

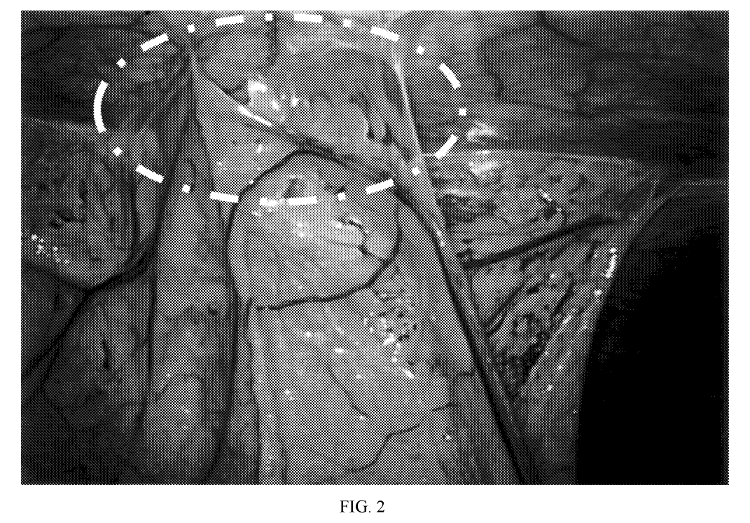

### DETAILED DESCRIPTION OF THE INVENTION

Unless otherwise defined, all terms, including technical and scientific terms, used herein have the same meaning as commonly understood by one having ordinary skill in the art to which this invention belongs. It is further understood that terms, such as those defined in commonly used dictionaries, should be interpreted as having a meaning that is consistent with their meaning in the context of the relevant art and the present disclosure and not be interpreted in an idealized or overly formal sense unless expressly so defined herein.

In describing the invention, it is understood that a number of techniques and steps are disclosed herein. Each of these has individual benefits and each can also be used in conjunction with one or more, or in some cases all, of the other disclosed techniques disclosed herein or otherwise known in the art. Accordingly, for the sake of clarity, this description refrains from repeating every possible combination of individual steps in an unnecessary fashion. Nevertheless, the specification and claims should be read with the understanding that such combinations are entirely within the scope of the invention and the claims.

The terminology used herein is for the purpose of describing particular embodiments only and is not intended to be limiting of the invention. As used herein, the term "and/or" includes any and all combinations of one or more of the associated listed items. As used herein, the singular forms "a," "an," and "the" are intended to include the plural forms as well as the singular forms, unless the context clearly indicates otherwise. It is further understood that the terms "comprises" and/or "comprising," when used in this specification, specify the presence of stated features, steps, operations, elements, and/or components, but do not preclude the presence or addition of one or more other features, steps, operations, elements, components, and/or groups thereof.

In the following description, for purposes of explanation, numerous specific details are set forth in order to provide a thorough understanding of the present invention. It is evident, however, to one skilled in the art that the present invention may be practiced without these specific details. The present disclosure is to be considered as an exemplification of the invention, and is not intended to limit the invention to the specific embodiments described below.

A new method and procedure is disclosed for treating any suitable adhesions in a mammalian body. The method and procedure can optionally be referred to as an adhesiolysis method and procedure. The invention can optionally include an apparatus, kit and system for performing some or all of the method and procedure of the invention. The adhesion to be treated can have undesirably bound together tissues that are not supposed to be connected to each other, for example organs to other organs, to omentum or to an abdominal wall or different loops of bowel. The adhesion to be treated can optionally include peritoneal adhesions, tendinous adhesions, pericardial adhesions, peritendinous adhesions, peridural adhesions or any combination of the foregoing, but is not limited thereto.

The method of the invention can optionally be part of a surgical procedure in a mammalian body. The method of the invention can optionally facilitate the surgical procedure, for example by obtaining or permitting access to a targeted area, which can optionally be called a surgical site, of the surgical procedure. The method of the invention can optionally inhibit or minimize damage, for example tearing or perforation, during the surgical procedure to the organs or other tissue bound to each other by the adhesion.

The treatment of the adhesion in the method of the invention can optionally include treating a portion or all of the adhesion. The treatment can optionally include breaking down, dissolving, lysing, separating or cutting the adhesion, or any combination of the foregoing, to permit access to the targeted surgical area or otherwise. The treatment can include softening or loosening the adhesion before mechanically or electrically lysing the adhesion or to completely lyse or treat the adhesion without the use of a mechanical or electrical tool or aid.

The method of the invention can include applying a material to all or a portion of the exposed surface of an adhesion, for example in the manner disclosed herein to treat the adhesion. The material can optionally be referred to as chemical aid, an adhesiolysis aid, a chemical adhesiolysis aid, an adhesiolysis solution or a chemical adhesiolysis solution. For simplicity, the treatment material of the invention is referred to herein as an adhesiolysis aid. The adhesiolysis aid can optionally include a collagenase-based component or material, which can optionally include at least one collagenase enzyme of any suitable type. The at least one collagenase enzyme can optionally be diluted in a solvent to form a solution. The concentration of the at least one collagenase enzyme in the adhesiolysis aid can optionally be calculated so as to inhibit or minimize any damage or effect of the adhesiolysis aid on nontargeted or adjoining tissue near the adhesion. The adhesiolysis aid, or such solution, can optionally include a buffer, stabilizer, emulsifier, a component that affects the consistency or viscosity of the solution or any combination of the foregoing. The adhesiolysis aid of the invention can optionally be in the form of a powder, foam, gel, cream, or paste, or soaked strip or mesh.

The adhesiolysis aid of the invention can optionally comprise a hemostatic agent, for example to address any bleeding caused by lysing vascularized adhesions. The amount of the collagenase enzyme in the adhesiolysis aid of the invention can vary, for example depending on the grade of the adhesions. For example, the amount of the collagenase enzyme can be increased as a function of the vascularization or density of the adhesion.

The adhesiolysis solution or aid can be delivered to the adhesions in any suitable manner, for example to the adhesion and optionally the area immediately around the adhesion as necessary to facilitate delivery of the adhesiolysis aid. The adhesiolysis aid can optionally be delivered in any suitable manner to a targeted area of the adhesion, wherein the “targeted area of the adhesion” is defined herein to mean the desired area of the adhesion for treatment and any immediately adjoining tissue or area necessitated for such practical targeted delivery of the adhesiolysis aid to the desired area, but excluding further area in the cavity area of the mammalian body in which the adhesion is located that would be affected by irrigating the adhesion or cavity, and excluding the entirety of the cavity area of the mammalian body in which the adhesion is located for example if such cavity area was washed with a material, solution or liquid. The adhesiolysis aid can optionally be selectively delivered to the adhesion for treatment, wherein “selectively delivered” and “selective delivery” are defined herein to mean delivery to the desired treatment area of the adhesion but not the entire cavity of the mammalian body in which the adhesion is located or the area of the cavity adjacent the adhesion that would be affected by irrigating the adhesion with a material, solution or liquid. The adhesiolysis aid can optionally be delivered directly to the adhesion, wherein “directly” is defined herein to mean a method in which the adhesiolysis aid makes contact with the desired area of the adhesion but not the entirety of the cavity of the mammalian body in which the adhesion is located, or the area of the cavity surrounding the adhesion that would be engaged by a material, solution or liquid irrigating the adhesion. The adhesiolysis aid can optionally be delivered to the adhesion, for example the treatment area of the adhesion or the targeted area of the adhesion, free of flooding or irrigating the body cavity in which the adhesion is located with the adhesiolysis aid, so that the adhesiolysis aid is not applied or delivered to the remainder of the body cavity or non-targeted tissue. The adhesiolysis aid can optionally be delivered to the adhesion, for example the treatment area of the adhesion or the targeted area of the adhesion, free of delivery to tissue adjoining or nearby the adhesion, so that the adhesiolysis aid is not applied or delivered to tissue adjoining or nearby the adhesion.

The method of the invention can optionally apply the adhesiolysis aid onto the treatment surface area of the adhesion, or the targeted area of the adhesion, as a powder, in which case the adhesiolysis aid is both selectively delivered to the adhesion and delivered directly to the treatment area of the adhesion. The method of the invention can optionally include spraying the adhesiolysis aid directly onto the treatment area of the adhesion, or the targeted area of the adhesion, for example as a liquid or foam, in which case the adhesiolysis aid is both selectively delivered to the adhesion and delivered directly to the treatment area of the adhesion. The method of the invention can optionally include delivering the adhesiolysis aid directly onto the treatment area of the adhesion, or the targeted area of the adhesion, by means of a syringe or other suitable selective tissue-targeting tool, device or applicator, in which case the adhesiolysis aid is both selectively delivered to the adhesion and delivered directly to the treatment area of the adhesion. The method of the invention can optionally include delivering the adhesiolysis aid to the treatment area of the adhesion, or the targeted area of the adhesion, as a foam, gel, cream, paste or other suitable applicator, in which case the adhesiolysis aid is both selectively delivered to the adhesion and delivered directly to the treatment area of the adhesion. The system can optionally include laying strips or meshes that are soaked with the adhesiolysis aid onto the treatment area of the adhesion, or the targeted area of the adhesion, in which case the adhesiolysis aid is both selectively delivered to the adhesion and delivered directly to the treatment area of the adhesion. These strips or meshes can be made out of a variety of suitable materials, including but not limited to bioresorbable materials. As stated above, the method of the invention can include at least one collagenase enzyme for treating an adhesion. The at least one collagenase enzyme can optionally be delivered onto a treatment surface area of the adhesion by means of a suitable applicator, which can include a spray device, a syringe, a foam, a gel, a cream, a paste, a powder, a strip or mesh or any combination of the foregoing. Such foam, gel, cream, paste, powder, strip or mesh can optionally be part of the adhesiolysis aid of the invention.

After application of the adhesiolysis aid to the treatment area of the adhesion, or the targeted area of the adhesion, the surgeon can optionally wait a period of time for the adhesiolysis aid to sufficiently treat the adhesion, for example sufficiently lyse the adhesion. The adhesiolysis aid can optionally be left on the selected portion of the adhesion for any suitable time period, for example not to exceed 19 minutes.

The method of the invention, for example utilizing the adhesiolysis aid of the invention, can optionally serve to soften and loosen adhesions before mechanically lysing the adhesion, electrically lysing the adhesion or both, or can optionally serve to completely lyse the adhesions without the use of any mechanical or electrical aid. The adhesiolysis aid of the invention can optionally completely or partially sever the adhesion. For example, mechanical adhesiolysis, electrical adhesiolysis or both can optionally be practiced after application of the adhesiolysis aid to the treatment area of the adhesion, or the targeted area of the adhesion, as needed. Use of such mechanical or electrical aids can optionally include pulling apart the softened or loosened adhesion so as to selectively lyse a portion or all of the adhesion.

The adhesiolysis aid of the invention may be applied to any portion of a mammalian or human body, for example to various intraperitoneal and intrathoracic viscera, epitendineum, gynecologic tissue, cardiac tissue, and to tendons and ligaments.

In one optional method of the invention, the adhesiolysis aid may be placed only on the densest adhesions, for example in any manner disclosed herein, before the surgeon starts lysing the lower grade adhesions. Such method would allow the adhesiolysis aid to act on the densest adhesions at the same time that the surgeon is breaking apart the lighter ones. By the time the surgeon finishes lysing the filmier adhesions, the denser adhesions will be chemically loosened, dissolved or treated, and can optionally be separated with a blunt or other tool as if they were filmy adhesions. In this manner, the overall time or length of the adhesiolysis procedure can be reduced.

The invention can optionally include kits that include the adhesiolysis aid of the invention, a device, applicator or tool for delivering the adhesiolysis aid in a suitable application or both. The kit of the invention can optionally be referred to as a chemical adhesiolysis kit. The kit or chemical adhesiolysis kit can optionally include the adhesiolysis aid, or chemical adhesiolysis aid, formulated as a powder, a spray or aerosol, a liquid, a foam, a gel, cream, or paste, a soaked strip or mesh or any combination of the foregoing. The kit or chemical adhesiolysis kit can optionally include the chemical adhesiolysis aid or product of the invention in a vial or other storage container from which the doctor can draw the product into an instrument not included in the kit to deliver the product onto the treated area, for example the targeted area of an adhesion in a manner disclosed herein. The delivery instrument can optionally be included in the kit. The kit or chemical adhesiolysis kit of the invention can optionally include the chemical adhesiolysis aid or product of the invention preloaded onto an instrument that will be used to deliver the product onto the treatment area, for example the targeted area of an adhesion in a manner disclosed herein. The kit or chemical adhesiolysis kit of the invention can optionally include the chemical adhesiolysis aid or product in a vial or other storage container and also include an instrument that will be used to deliver the product onto the treatment area, for example the targeted area of an adhesion in a manner disclosed herein.

The kit or chemical adhesiolysis kit of the invention can optionally include the chemical adhesiolysis aid or product as a liquid to be soaked onto strips or meshes not included in the kit. The kit or chemical adhesiolysis kit of the invention can optionally include strips or meshes that have been pre-soaked in the chemical adhesiolysis aid or product. The kit or chemical adhesiolysis kit of the invention can optionally include the chemical adhesiolysis aid product and also include strips or meshes to soak in the aid product before applying to the treatment area, for example the targeted area of an adhesion in a manner disclosed herein.

The kit or chemical adhesiolysis kit of the invention can optionally include a second product, which can optionally be referred to as an inhibitory product, that slows, stops, or otherwise inhibits the activity of the chemical adhesiolysis aid or product. The inhibitory product can optionally be applied onto the treated treatment area, or the targeted area of an adhesion, as a powder. The inhibitory product can optionally be sprayed or poured onto the treatment area, or the targeted area of an adhesion, for example as a liquid. A liquid inhibitory product can optionally be applied to the adhesion by a suitable delivery tool, for example a syringe or other suitable tool, device or applicator, that engages the adhesion when delivering the liquid to the adhesion. The solution can optionally be applied to the treatment area, or the targeted area of an adhesion, as a foam, gel, cream, paste or other suitable applicator. The inhibitory product can optionally include laying strips or meshes that are soaked with the solution onto the treatment area, or the targeted area of an adhesion. These strips or meshes can be made out of a variety of suitable materials, including but not limited to bioresorbable materials.

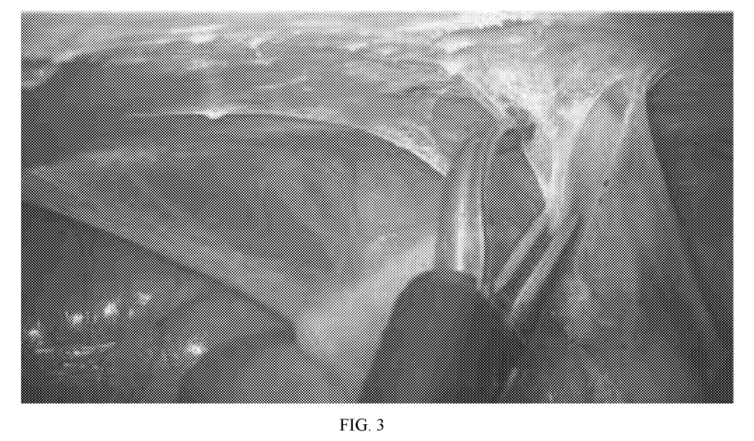

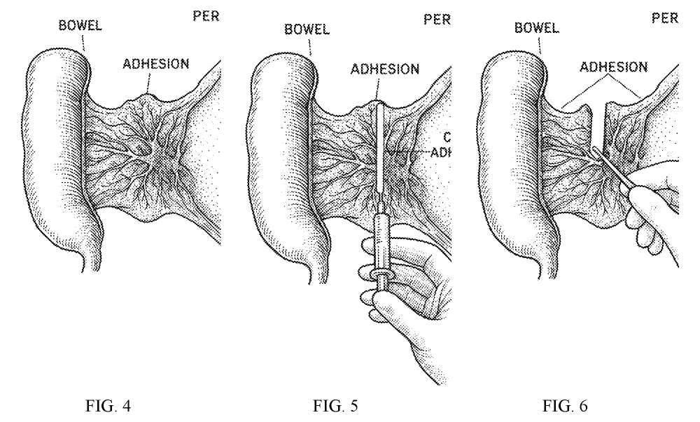

The method of the invention can optionally be used in any suitable procedure, for example laparoscopic procedures, open procedures or both. An optional method of the invention uses an adhesiolysis aid of the invention, for example a chemical adhesiolysis gel, in revision gastrointestinal surgery (see FIGS. 4-6). A surgeon begins the revision gastrointestinal surgery on a patient and discovers that the patient has adhesions connecting the bowel to the peritoneum. Due to the adhesions, the surgeon cannot easily proceed to the surgical site and begin the operation. The surgeon applies the adhesiolysis aid to the targeted area of the adhesion, for example in any of the methods disclosed herein. For example, the surgeon optionally loads a syringe with the chemical adhesiolysis aid and applies it to the adhesion, or to the targeted area of the adhesion. The surgeon optionally selectively delivers the chemical adhesiolysis aid to the targeted area of the adhesion (see FIG. 5). The chemical adhesiolysis aid loosens or otherwise treats the adhesion. The surgeon optionally separates the loosened or treated area of the adhesion in any suitable manner, for example with a blunt tool like a surgical probe. The method of the invention obviates the need to cut the adhesion apart with surgical scissors or tools, thus reducing the risk of injury to adjacent tissues or organs of the patient.

A kit comprising of a chemical adhesiolysis aid can optionally be provided where the chemical adhesiolysis aid is formulated as a powder, a spray or aerosol, a liquid or foam, a gel, cream, or paste, a soaked strip or mesh, or any combination of the foregoing. The kit can optionally include an inhibitory product that slows, stops, or otherwise inhibits the activity of the chemical adhesiolysis aid. The inhibitory product can optionally be formulated as a powder, a spray or aerosol, a liquid or foam, a gel, cream, or paste, a soaked strip or mesh, or any combination of the foregoing.

A chemical adhesiolysis aid formulated as a gel can optionally be provided. The gel can optionally comprise one or more collagenase enzymes.

The present invention provides a novel method for reducing injuries from adhesiolysis by chemically loosening or lysing adhesions. The method of the invention can optionally take surgical blades out of the adhesiolysis workflow so as to minimize the risk of cutting the tissue of the body joined by the adhesion.

I claim:

1. A surgical procedure in a body of a patient having an adhesion bound to adjoining tissue in the body, comprising selectively delivering a chemical adhesiolysis material to a treatment area of the adhesion, waiting a period of time for the chemical adhesiolysis material to sufficiently loosen the adhesion and performing the surgical procedure at a surgical site in the body, the loosening of the adhesion with the chemical adhesiolysis material permitting access to the surgical site and reducing the risk of damage to the adjoining tissue in the body.

2. The method of claim 1, wherein the chemical adhesiolysis material comprises at least one collagenase enzyme.

3. The method of claim 2, wherein the at least one collagenase enzyme is diluted in a solvent.

4. The method of claim 1, wherein the chemical adhesiolysis material includes a component selected from the group consisting of a buffer, a stabilizer, an emulsifier, a component that affects the consistency or viscosity of the chemical adhesiolysis material, a hemostatic agent and any combination of the foregoing.

5. The method of claim 1, wherein the selectively delivering step is selected from the group comprising of delivering the chemical adhesiolysis material to the treatment area as a powder, spraying the chemical adhesiolysis material onto the treatment area, delivering the chemical adhesiolysis material directly to the treatment area as a liquid or foam, delivering the chemical adhesiolysis material to the treatment area as a foam, gel, cream, or paste, delivering the chemical adhesiolysis material to a treatment area by means of a strip containing a collagenase solution and any combination of the foregoing.

6. The method of claim 1, wherein the waiting step does not exceed 19 minutes.

7. The method of claim 1, wherein a surgical procedure is selected from the group consisting of a laparoscopic procedure and an open procedure.

8. The method of claim 1, further comprising the step of applying an inhibitory product to the chemical adhesiolysis material on the treatment area to inhibit the effect of the chemical adhesiolysis material.

9. The method of claim 8, wherein the inhibitory product is applied using a method selected from the group comprising of applying the inhibitory product onto the treatment area as a powder, spraying the inhibitory product onto the treatment area, applying the inhibitory product onto the treatment area as a liquid or foam with a delivery tool that engages the treatment area, applying the inhibitory product to the treatment area as a foam, gel, cream, or paste, applying the inhibitory product to the treatment area by means of a strip or mesh containing the collagenase solution that is placed on the treatment area and any combination of the foregoing.

10. The method of claim 1, wherein the adhesion is in a body cavity and the selectively delivering step includes selectively delivering the chemical adhesiolysis material to the treatment area of the adhesion free of flooding or irrigating the entire cavity or treatment area with the chemical adhesiolysis material.

11. A surgical procedure in a body of a patient having an adhesion bound to adjoining tissue in the body, comprising delivering a chemical adhesiolysis material to a treatment area of the adhesion free of delivery of the chemical adhesiolysis material to the adjoining tissue in the body, waiting a period of time for the chemical adhesiolysis material to sufficiently loosen the adhesion and performing the surgical procedure at a surgical site in the body, the lysing of the adhesion with the chemical adhesiolysis material permitting access to the surgical site and reducing the risk of damage to the adjoining tissue in the body.

12. The method of claim 11, wherein the chemical adhesiolysis material comprises at least one collagenase enzyme.

13. The method of claim 11, wherein the chemical adhesiolysis material includes a component selected from the group consisting of a buffer, a stabilizer, an emulsifier, a component that affects the consistency or viscosity of the chemical adhesiolysis material, a hemostatic agent and any combination of the foregoing.

14. The method of claim 11, wherein the delivering step is selected from the group comprising of delivering the chemical adhesiolysis material to the treatment area as a powder, spraying the chemical adhesiolysis material onto the treatment area, delivering the chemical adhesiolysis material directly to the treatment area as a liquid or foam, delivering the chemical adhesiolysis material to the treatment area as a foam, gel, cream, or paste, delivering the chemical adhesiolysis material to a treatment area by means of a strip or mesh containing a collagenase solution and any combination of the foregoing.

15. The method of claim 11, further comprising the step of applying an inhibitory product to the chemical adhesiolysis material on the treatment area to inhibit the effect of the chemical adhesiolysis material.

16. A surgical procedure in a body of a patient having an adhesion bound to adjoining tissue in the body, comprising delivering at least one collagenase enzyme onto a treatment surface area of the adhesion by means of an applicator, the applicator being selected from the group consisting of a spray device, a syringe, a foam, a gel, a cream, a paste, a powder, a strip, a mesh and any combination of the foregoing, the lysing of the adhesion with the at least one collagenase enzyme permitting access to the surgical site and reducing the risk of damage to the adjoining tissue in the body.

17. The method of claim 16, wherein the at least one collagenase enzyme is diluted in a solvent.

18. The method of claim 16, wherein the at least one collagenase enzyme is mixed with a component selected from the group consisting of a buffer, a stabilizer, an emulsifier, a component that affects the consistency or viscosity of the at least one collagenase enzyme, a hemostatic agent and any combination of the foregoing.

19. The method of claim 16, further comprising the step of applying an inhibitory product to the at least one collagenase enzyme on the treatment area to inhibit the effect of the at least one collagenase enzyme.
